(19) United States
(12) Patent Application Publication
(10) Pub. No.: US 2011/0027880 A1
(43) Pub. Date: Feb. 3, 2011

(54) CELL CULTURE SYSTEM FOR PANCREATIC ISLANDS

(75) Inventors: Wendy Margaret MacFarlane, West Sussex (GB); Moira Harrison, East Sussex (GB); Claire Elizabeth Marriott, Sussex (GB)

Correspondence Address: VOLPE AND KOENIG, P.C., UNITED PLAZA, 30 SOUTH 17TH STREET, PHILADELPHIA, PA, 19103, US

(73) Assignee: UNIVERSITY OF BRIGHTON, Sussex (GB)

(21) Appl. No.: 12/812,775

(22) PCT Filed: Jan. 14, 2009
(86) PCT No.: PCT/GB09/50024

§ 371 Date: Sep. 27, 2010

(30) Foreign Application Priority Data
Jan. 14, 2008 (GB) 0800524.1

(51) Int. Cl. C12N 5/071

(52) U.S. Cl. 435/366

(57) ABSTRACT

Three-dimensional (3D) insulin-producing cell clusters derived from stem cells (preferably human embryonic stem cells) are provided by this invention, together with a method for their production using a microgravity bioreactor cell culture system.

This invention relates to three-dimensional (3D) insulin-producing cell clusters derived from stem cells and a method for their production using a microgravity bioreactor cell culture system. It can be performed using established human stem cell lines and without using and destroying human embryos.

### Regulation of Blood Glucose

Blood glucose is regulated through release of insulin from the pancreas. Insulin facilitates the passage of glucose from the bloodstream into cells, where it can be utilised as a vital energy source. In patients with diabetes, there is either a complete absence of insulin (Type 1 diabetes) or a problem in utilising insulin (Type 2 diabetes).

Insulin is produced by the beta cells within the pancreas, which are contained within ball-like structures called the islets of Langerhans. Beta cells (β-cells) are highly sensitive to changes in blood glucose and secrete active (fully processed) insulin in response to elevated blood glucose. Islets also contain other cell types which produce other hormones, such as glucagon (produced by alpha cells). The beta cells are held tightly in a 3-dimensional ball, surrounded by alpha cells (α-cells) and, importantly, benefiting from an excellent blood supply.

The three-dimensional structure of a pancreatic islet ensures that there is direct cell-cell contact enabling beta cells to communicate with their neighbouring cells. Such structure and communication is vital for the exquisite control of blood glucose required to maintain a healthy functioning organism. Not only are the cells packed together in an islet, the islet itself is held within the structure of the pancreas. In order to maintain physiological function, beta cells need to be held together and to benefit from good oxygen delivery, nutrient delivery and removal of waste products generated by cell respiration. This is ensured by the highly vascularised nature of a pancreatic islet.

Although the discovery and isolation of insulin in the early 20^{th} century ensured that people with diabetes did not die, over 80 years of clinical practice has shown that insulin injections are not a cure for diabetes. This is because injection of exogenous insulin cannot mimic the very tight blood glucose regulation that an islet can exhibit. As a result of a failure to accurately control blood glucose concentrations, diabetes patients using insulin injections still develop a range of serious health problems, with diabetes being the leading cause of heart disease, kidney failure, blindness and lower limb amputation in the western world.

### Current Treatment Regimes

The number of people with diabetes has reached epidemic proportions worldwide. It is estimated that within the next 20 years, 30 million will be affected by Type 1 diabetes and 300 million with Type 2 diabetes. Type 1 diabetes is usually diagnosed in childhood or as a young adult. Without insulin it is rapidly fatal. Although Type 2 diabetes can usually be treated with diet and tablets at first, most people eventually need insulin treatment. Diabetes massively increases the risk of dying from a heart attack or stroke and remains a leading cause of blindness, kidney failure and leg amputation. These complications, which account for 10% of the annual NHS budget in the UK, can be prevented by keeping glucose levels as normal as possible. Achieving this with conventional insulin treatment requires continuous hard work, multiple daily insulin injections and frequent painful finger-prick glucose testing. Even with all this effort, dangerously low blood glucose levels are common. Every insulin injection is associated with a risk of low blood glucose (hypoglycaemia) which can result in collapse without warning. The risk of severe hypoglycaemia is one of the greatest fears for those with diabetes and is an ongoing burden for all on insulin therapy. It may restrict employment and lead to loss of quality of life.

In 2000, James Shapiro and colleagues in Edmonton, Canada confirmed that liberation from insulin injections and freedom from hypoglycemia with virtually normal glucose levels could be achieved in Type 1 diabetes following isolation and purification of the insulin-secreting islet cells from a post-mortem donor pancreas. These clusters of insulin-producing cells, which make up only 1-2% of whole pancreas, are transplanted directly into the portal vein which drains into the liver. This is performed through the skin using a simple syringe injection mechanism under X-ray guidance with only a local anaesthetic. This procedure offers a future without insulin injections for everyone with diabetes. However, this will not be possible unless a new source of insulin-producing cells is found. It is hard to purify enough islets for success from a single donor. Often only 50% of islet preparations are suitable for clinical transplantation and up to four transplants, each requiring a separate donor, have been required for a single individual with Type 1 diabetes to stop insulin injections. It has been estimated that existing rates of organ donation would be enough to transplant less than 1% of people with Type 1 diabetes. So, although it is a promising technology, availability remains a limiting factor.

Scientific efforts have concentrated on trying to develop sources of insulin-producing cells (e.g. from stem cells) and looking at ways of delivering them into recipients. Even if it was possible to produce an insulin-producing cell from a stem cell, the problem still remains as to how to introduce that cell into a recipient. Many different avenues for delivery have been explored around the world, most focusing on some kind of encapsulation in an artificial substrate such as alginates or polymers. To date there has been no truly successful method of encapsulation. Encapsulation is limited by the need to ensure oxygen and nutrient diffusion to cells as well as allowing insulin to leave the cells.

The present inventors have sought to provide a solution to these problems by applying their knowledge of stem cell biology to drive the formation of new insulin-producing cells from stem cells, and also by combining this with a unique microgravity cell culture environment. Together, this will allow the generation of transplantable 3D insulin-producing cell clusters which function like normal islets, and which can be transplanted into patients using the same transplant techniques currently being employed to deliver donated islets.

### Traditional Cell Culture Methods

Traditional cell culture methods in laboratories around the world involve the growing of cells on coated plastic flasks which produces confluent flat monolayers of cells with a two-dimensional sheet-like structure. Cells grown this way do not experience three-dimensional cell contact like cells in the body and as a result often behave in a non-physiological manner. This can involve a loss of sensitivity to important stimuli, or, commonly, a loss of control of growth resulting in continued proliferation. In terms of clinical transplantation, uncontrolled growth is unacceptable as this poses the risk of tumour formation upon transplantation.

Given the limitations and risks associated with the growth of cells in traditional 2D cell culture flasks, the present inventors have developed their novel approach utilising a 3D microgravity cell culture environment known as a bioreactor.

### Bioreactors

By definition, a bioreactor is a device that supports a biologically active environment in which cells or microorganisms (bacteria) can be grown. Normally, these cells are grown in very large bioreactors in order to harvest a product they have produced (or secreted), such as antibodies or proteins. The cells and their products can be grown and harvested on an industrial scale and under optimum conditions. The environment within the bioreactor, such as gases (oxygen, nitrogen and carbon dioxide), temperature and pH can be very strictly controlled. Agitation of the bioreactor is essential, but traditional methods involve physical stirring of the contents. This can be extremely damaging to the cells as they can experience strong shear forces and foaming. Bioreactors are not normally suitable for adherent cell or tissue culture.

NASA scientists recently developed a revolutionary rotating microgravity bioreactor that creates an environment for cells which mimics that in the human body. It was created to allow scientists to perform cell culture in space, as the spinning vessel neutralizes gravitational influences and encourage cells to grow in a natural manner. Ground tests of the bioreactor yielded three-dimensional tissue specimens with approximating natural growth, a striking change from the pancake shapes of traditional adherent cell cultures. In humans and other animals, as cells replicate, they “self associate” to form clusters held together by a complex matrix made up of collagens, proteins, fibres and other chemicals. This highly evolved microenvironment informs neighbouring cells how they should grow, into what shapes, and how to respond to stimuli. Thus, they afford the opportunity to study the complex order of tissue, and to generate physiologically accurate tissue, in a culture system that can be manipulated by drugs, hormones and tightly controlled nutritional conditions.

Studying these mechanisms outside the body on Earth is limited by the effects of gravity, as cells do not easily self-associate to grow naturally. The bioreactor promotes self-association in a container about the size of a soup can. It is composed of a clear shell and the centre holds a cylindrical filter that passes oxygen and nutrients in and allows carbon dioxide and waste out. This ensures that the fluid rotates without shear forces that would destroy the cells. Strictly speaking, the rotating vessel does not actually cancel gravity, but ideally maintains cells in continual free-fall similar to that experienced by astronauts in the microgravity of space.

The present inventors have utilised this bioreactor to culture insulin-secreting cells (which would normally be grown in a monolayer) in order to allow them to grow and aggregate into an islet-like cluster and thus recreate the environment that they would naturally occur in.

As described above, delivering whole donated islets into the portal vein (liver) is a successful transplantation strategy that can reverse diabetes. This procedure is cripplingly limited by donor availability and in addition needs intense immune suppression due to the multiple donors required. There is therefore a need to “manufacture” physiologically identical islets in large quantities that could be delivered in a similar way to primary islet transplants and which would be derived from a single source, enabling immune suppression to be minimised. The present invention seeks to meet that need.

WO 2007/075807 relates to methods for the directed differentiation of embryonic stem cells. Examples 7 to 18 describe the use of human embryonic stem cells to generate embryonic bodies. The resulting groups of cells express pdx-1 and are suspended in a commercially produced gel. They are not grown in a microgravity environment.

WO 2004/007683 discloses techniques for inducing the differentiation of progenitor cells or stem cells. Example 8 describes the differentiation of liver progenitor cells (not stem cells) into insulin-secreting beta-like cells. The liver cells are not grown in a microgravity environment; they are grown in hydrogel.

In Vitro Cell. Dev. Biol.-Animal; Vol 37, pp 490-498 (2001), Cameron et al., “Formation of insulin-secreting, sertoli-enriched tissue constructs by microgravity co-culture of isolated pig islets and rat sertoli cells” describes work relating to the culture of pig and rat cells in a microgravity environment and using a commercially available gel. There is no teaching or suggestion of the use of human cells, and in particular no mention of human embryonic stem cells, nor of the unique and highly specialised differentiation protocol that would be required for growing them in a microgravity environment.

Ann. NY Acad. Sci, Vol 944, pp 420-428 (2001), Cameron et al., “Formation of sertoli cell-enriched tissue constructs utilizing simulated microgravity technology” once again relates to the co-culture of rat and pig cells based on the use of a commercially available gel. There is no teaching or suggestion of the use of human cells.

WO 97/16536 refers to methods for the ex vivo proliferation and differentiation of neonatal and/or adult human or non-human pancreatic islets. The cells are cultured in a microgravity environment and an aggregation medium is employed. The intention is to produce products useful for the treatment of diabetes. It is important to note that WO 97/16536 describes work with adult human tissue and not embryonic stem cells. The tissue used is donated pancreatic cells from cadavers. The work involves disaggregating the pancreatic islets and then reassembling them using a culture system, and there is a proposal to co-culture with other adult cell types.

In contrast to the previous approaches reviewed above, the present invention takes stem cells from a single source, generates from them functional insulin-producing cells and then promotes formation of transplantable clusters of these newly formed cells. It uses pure, undifferentiated human stem cell lines as a starting material. The inventors use a unique starting material, have developed a unique differentiation protocol (for converting stem cells to insulin-producing cells) and, consequently, have a new method in the combination of these with their optimised microgravity bioreactor culture system. It is important to realise that the clustering of human embryonic stem-cell derived insulin-producing cells has not previously been achieved.

According to the present invention there is provided three-dimensional insulin-producing cell clusters derived from stem cells. The invention further provides a method for producing three-dimensional insulin-producing cell clusters derived from stem cells in a microgravity environment. More particularly, the method involves the use of a microgravity bioreactor culture system.

In a specific embodiment, the method of this invention comprises initially culturing stem cells on a static plate or dish in a medium that promotes the formation of embryoid bodies, and subsequently transferring the cells to a microgravity bioreactor where they are cultured in a series of media such that the cells firstly form 3D clusters of embryoid bodies and then further differentiate into insulin-producing beta cells.

In a particularly preferred embodiment, the method of this invention comprises the following steps:—
a) initially culturing stem cells on a static plate or dish in glucose DMEM supplemented with foetal bovine serum for a period of four days;
b) transferring them to a microgravity bioreactor and culturing under rotation for three days;
c) replacing the culture medium by serum-free DMEM/F12 with insulin-transferrin-selenium supplement and culturing for a further four days;
d) selecting nestin positive cells by gravity sedimentation and incubating them in the microgravity bioreactor with DMEM/F12 media supplemented with insulin, transferrin, progesterone, sodium selenite, human keratinocyte growth factor, epidermal growth factor, B27 supplement and nicotinamide for seven days; and
e) recovering 3D insulin-producing cell clusters.

The inventors have developed a technique that allows individual beta cells to form their own physiologically correct, 3D islet structure by culturing them in a microgravity bioreactor. Configuring beta cells in this way enhances glucose-stimulated insulin release, maintains cell viability (even at the centre of the ball of cells) and allows islets to be “grown” in large numbers and maintained over extended periods.

The inventors have optimised their protocol to generate insulin-producing cells from embryonic stem cells. They have generated the first ever 3D clusters of cells from stem cells using their microgravity bioreactor based system. They have proof of principle from islet transplants that 3D clusters of cells can be safely delivered into the hepatic portal vein of patients with diabetes. Generation of insulin-producing cell clusters from stem cells could allow the creation of a potentially limitless supply of transplantable cells for all patients with diabetes. The market for glucose-responsive insulin-producing cell clusters produced from their optimised bioreactor system is therefore enormous.

The formation of insulin-producing cells from stem cells using the method of the present invention has never previously been attempted. The method uses a protocol which involves the initial treatment of the stem cells with an optimised cocktail of stimuli known to drive the formation of beta cells during normal development. At day 5 of the protocol, cells are transferred to the microgravity bioreactor. At this stage, the cells form 3D clusters called embryoid bodies (EBs) and begin down the path towards becoming a beta cell. Initial results suggest that EBs formed and maintained in the bioreactor system of this invention are superior in structure and stability to those formed by traditional cell culture methods. Following further defined changes in the nutritional and hormonal environment within the bioreactor vessels, the EBs are driven to form insulin-producing cells. The first 3D cell clusters have been generated in the optimised bioreactor system.

The present invention will now be illustrated by the following Example and with reference to the accompanying FIGURE which shows:—

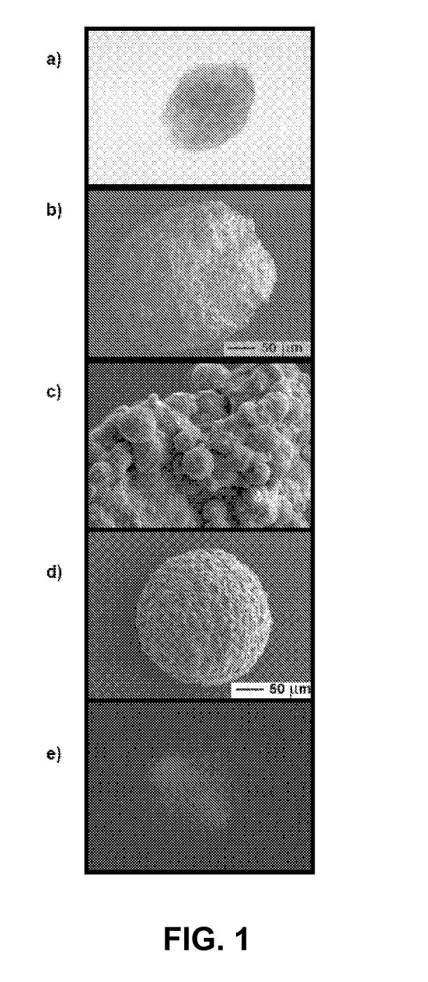

FIG. 1:
a) Primary mouse islet;
b) Scanning electron microscope image of a bioreactor-cultured islet created from MIN6 insulin-secreting cells;
c) Scanning electron microscope image of the surface structure of a bioreactor-cultured islet created from MIN6 insulin-secreting cells;
d) Superior embryoid body (EB) generated from stem cells using the bioreactor culture method described herein;
e) Insulin production from a 3D cluster of insulin-secreting cells generated from stem cells using the bioreactor culture method described herein.

### EXAMPLE

### Protocol for the Generation of Differentiated Insulin Secreting Clusters (DISCs)

Formation of embryoid bodies (EBs) from the starting stem cell population was directed by culture initially in Petri dishes in 25 mM glucose DMEM supplemented with 10% foetal bovine serum. Following four days in dish culture, EBs were transferred to a 10 ml HARV (High Aspect Rotating Vessel) microgravity bioreactor vessel (supplied by Synthecon) and cultured at a rotating speed of ˜12 rpm for 3 days. After this time media was replaced with serum-free DMEM/F12 with insulin-transferrin-selenium supplement. EBs were cultured in the microgravity HARV for 4 days. Nestin positive cells were selected by gravity sedimentation and replaced into the HARV wherein they were incubated with DMEM/F12 media supplemented with 25 μg/ml insulin, 100 μg/ml transferrin, 20 nM progesterone, 30 nM sodium selenite, 10 ng/ml human keratinocyte growth factor, 20 ng/ml epidermal growth factor, B27 supplement and 10 ng/ml nicotinamide for 7 days at a rotating speed of ˜12 rpm. Samples were taken at 7 days; cells were transferred to a tissue culture treated 4-well chamber slide, allowed to attach for 24 hours and then fixed in 3.7% formalin prior to ICC staining.

FIG. 1 illustrates a) a primary islet isolated from a mouse, b) a scanning electron microscope image of an islet generated from insulin-producing cells using bioreactor culture, c) a scanning electron microscope image of the surface of the same islet, d) a scanning electron microscope image of an embryoid body (EB) produced according to the protocol described in this Example, and e) insulin production from a 3D insulin-producing cell cluster generated from stem cells using the protocol described in this Example. It is clear from these images that culturing clusters of insulin-producing cells in the bioreactor has allowed them to self-associate and indeed form a complex matrix which is indistinguishable from that of the primary islet.

1. A three-dimensional insulin-producing cell cluster derived from stem cells.

2. A three-dimensional insulin-producing cell cluster as claimed in claim 1, wherein the stem cells from which it is derived are human embryonic stem cells.

3. A three-dimensional insulin-producing cell cluster as claimed in claim 1, wherein the cell cluster is produced in a microgravity environment.

4. A three-dimensional insulin-producing cell cluster as claimed in claim 3, wherein the microgravity environment is a microgravity bioreactor cell culture system.

5. A method for producing three-dimensional insulin-producing cell clusters derived from stem cells and which comprises the use of a microgravity environment.

6. A method as claimed in claim 5, wherein the microgravity environment comprises a microgravity bioreactor cell culture system.

7. A method as claimed in claim 5, which comprises initially culturing stem cells on a static plate or dish in a medium that promotes the formation of embryoid bodies, and subsequently transferring the cells to a microgravity bioreactor where they are cultured in a series of media such that the cells firstly form 3D clusters of embryoid bodies and then further 30 differentiate into insulin-producing beta cells.

8. A method as claimed in claim 5, which comprises the following steps:
a) initially culturing stem cells on a static plate or dish in glucose DMEM supplemented with foetal bovine serum for a period of four days;
b) transferring them to a microgravity bioreactor and culturing under rotation for three days;
c) replacing the culture medium by serum-free DMEM/F12 with insulin-transferrin-selenium supplement and culturing for a further four days;
d) selecting nestin positive cells by gravity sedimentation and incubating them in the microgravity bioreactor with DMEM/F12 media supplemented with insulin, transferrin, progesterone, sodium selenite, human keratinocyte growth factor, epidermal growth factor, B27 supplement and nicotinamide for seven days; and
e) recovering 3D insulin-producing cell clusters.

9. A method as claimed in claim 5, wherein the stem cells are human embryonic stem cells.
